(19) United States
(12) Patent Application Publication
(10) Pub. No.: US 2016/0279036 A1
(43) Pub. Date: Sep. 29, 2016

(54) FOAM-STABILIZED HAIR DYE

(71) Applicant: Henkel AG & Co. KGaA, Duessedorf (DE)

(72) Inventors: Juergen Schoepgens, Schwalmtal (DE); Burkhard Mueller, Duesseldorf (DE)

(73) Assignee: Henkel AG & Co. KGaA, Duesseldorf (DE)

(21) Appl. No.: 15/175,963

(22) Filed: Jun. 7, 2016

Related U.S. Application Data
(63) Continuation of application No. PCT/DE2014/200500, filed on Sep. 23, 2014.

(30) Foreign Application Priority Data
Dec. 17, 2013 (DE) 10 2013 226 283.3

(51) Int. Cl. A61K 8/04; A61K 8/41; A61K 8/36; A61K 8/73; A61Q 5/10; A61K 8/22

(52) U.S. Cl. CPC A61K 8/046; A61Q 5/10; A61K 8/22; A61K 8/361; A61K 8/73; A61K 8/41; A61K 2800/87; A61K 2800/4324

(57) ABSTRACT

An agent for coloring keratin fibers includes at least two preparations (A) and (B) packaged separately from one another, and optionally a further preparation (C) packaged separately from (A) and (B), which are mixed directly prior to application to provide an application mixture. Preparation (A) includes at least one oxidation dye precursor and at least one alkali metal salt of at least one fatty acid, and preparation (B) is flowable and includes, based on its weight, at least one oxidant and xanthan gum. The agent leads to extremely stable foams which allow the simple and intensive coloration of fibers. The stability is retained even in the presence of high concentrations of salt.

### FIELD OF THE INVENTION

The present invention generally relates to agents for coloring which are to be applied by special application devices in the form of a stable foam, to a coloring method involving the use of the agents, and to the application device, as well as to a corresponding kit for coloring keratin-containing fibers.

### BACKGROUND OF THE INVENTION

In general, either direct dyes or oxidation dyes, which are formed by the oxidative coupling of one or more developer components to one another or with one or more coupler components, are used for coloring keratin-containing fibers. Coupler and developer components are also called oxidation dye precursors. Primary aromatic amines with a further free or substituted hydroxy or amino group, located in the para or ortho position, diaminopyridine derivatives, heterocyclic hydrazones, 4-aminopyrazolone derivatives, and 2,4,5,6-tetraaminopyrimidine and derivatives thereof are typically used as developer components. m-Phenylenediamine derivatives, naphthols, resorcinol and resorcinol derivatives, pyrazolones, m-aminophenols, and substituted pyridine derivatives are normally used as coupler components.

In order to stabilize the dye precursors during storage and to accelerate the reaction during the oxidative application, oxidation dyes mostly have an alkaline pH, which is adjusted with alkalizing agents, such as alkanolamines, ammonia, or inorganic bases. Although in this regard ammonia in particular enables good coloring results, it also manifests disadvantages for the user due to its odor and irritation potential for skin and mucous membranes.

Oxidation dyes usually consist of two components whose mixture is sufficiently viscous to allow it to be applied comfortably to hair without any dripping or running. Moreover, efforts have often been made to develop other presentation forms. Thus, it was proposed to apply lower viscosity dyes to hair with special applicator systems or to apply dyes as a foam. The use of aerosol foams in particular is widespread for foam application.

There has also been a requirement recently, however, to be able eliminate the use of propellant gases. A further problem in foam application is the stabilization of the foam. The quality of foams is considered to be ideal when dispensing of the product affords a solid, stable foam that leaves a supple feel and breaks down only slowly on the hair. It is frequently observed, however, that the applied foams possess little stability and rapidly collapse again, leaving behind a low-viscosity solution that drips. In another respect, it is also essential that the foam nevertheless wets the hair well so that a good color application can be realized. Foam stability is negatively influenced in particular by the presence of larger amounts of salts and dye (precursor)(s). Hair treatment agents in the form of foams are already known from JP10-167938 A; it discloses betaine surfactants of the alkyl dimethyl betaine type and (fatty acid amidoalkyl)dialkyl betaine type, such as cocoamidopropyl betaine, as especially suitable surfactants for a stable foam formation of acidic, hydrogen peroxide-containing hair treatment agents. Cocoamidopropyl betaine is also described in WO 2006/066642(A1) as a suitable surfactant with high surfactant content in tinting foams based on direct dyes.

However, it has been shown in extensive tests that foam hair dyes pose particular challenges to the formulation skills of the product developer. Thus, on the one hand, the foam must be stable enough to assure problem-free use, but, on the other, it must break down rapidly during application in order to assure a uniform and intense wetting of the hair.

If the foam nevertheless breaks down too rapidly, then the liquid application mixture flows from the hair and from the scalp onto the forehead, neck, and temple area and there results in annoying discolorations of the skin or clothing or even to irritation of the eyes.

It is therefore desirable to optimize oxidation dyes for foam application without use of propellant gases, so that the aforesaid disadvantages can be overcome. In particular, stable dye foams without leakage problems should to be provided.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An agent for coloring keratin fibers includes at least two preparations (A) and (B), packaged separately from one another, and optionally a further preparation (C) packaged separately from (A) and (B), which are mixed immediately before use to form an application mixture, wherein preparation (A) includes at least one oxidation dye precursor and at least one alkali metal salt of at least one fatty acid; and preparation (B) is flowable and, based on its weight, includes at least one oxidizing agent and xanthan gum.

Also provided is a method for coloring keratin fibers, in which at least two preparations (A) and (B), packaged separately from one another, and optionally a further preparation (C), packaged separately from (A) and (B), are mixed immediately before use to form an application mixture and are applied from a dispenser in the form of a foam, characterized in that preparation (A) includes at least one oxidation dye precursor and at least one alkali metal salt of at least one fatty acid; and preparation (B) is flowable and, based on its weight, includes at least one oxidizing agent and xanthan gum.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was found surprisingly that the aforesaid problems can be resolved by special oxidation dyes. The stability is retained even in the presence of higher salt concentrations. In addition, the agents of the invention enable more intensive coloring results and significantly longer-lasting coloring results than previously known foam preparations. Lastly, the agents are low in odor.

The present invention firstly constitutes agents for coloring keratin fibers, including at least two preparations (A) and (B), packaged separately from one another, and optionally a further preparation (C), packaged separately from (A) and (B), which are mixed immediately before use to form an application mixture, wherein

i. preparation (A) includes
a) at least one oxidation dye precursor and
b) at least one alkali metal salt of at least one fatty acid and

ii. preparation (B) is flowable and, based on its weight, includes
a) at least one oxidizing agent and
b) xanthan gum.

Second, the present invention includes methods for coloring keratinic fibers, in which at least two preparations (A) and (B), packaged separately from one another, and optionally, a further preparation (C), packaged separately from (A) and (B), are mixed immediately before use to form an application mixture and are applied from a dispenser in the form of a foam, wherein

i preparation (A) includes
a) at least one oxidation dye precursor and
b) at least one alkali metal salt of at least one fatty acid
   and

ii. preparation (B) is flowable and, based on its weight, includes
c) at least one oxidizing agent and
d) xanthan gum.

Keratin-containing fibers are understood to be wool, pelts, feathers, and particularly human hair. The dyes of the invention can also be used in principle for coloring other natural fibers, however, such as, e.g., cotton, jute, sisal, linen, or silk, modified natural fibers such as, for example, regenerated cellulose, nitrocellulose, alkyl or hydroxyalkyl cellulose, or acetyl cellulose.

The agents of the invention include the active substances in a cosmetically acceptable carrier. Said cosmetic carrier is preferably aqueous, alcoholic, or aqueous-alcoholic. An aqueous carrier in the context of the invention includes at least 40% by weight, particularly at least 50% by weight of water. Aqueous-alcoholic carriers in the context of the present invention are understood to be water-containing compositions including 3 to 70% by weight of a C₁-C₄ alcohol, particularly ethanol or isopropanol. The agents of the invention can also include in addition further organic solvents such as, for example, 4-methoxybutanol, ethyl diglycol, 1,2-propylene glycol, n-propanol, n-butanol, n-butylene glycol, glycerol, diethylene glycol monoethyl ether, and diethylene glycol mono-n-butyl ether, in so far as they do not have an excessive negative effect on foam formation and foam stability. In this case, all water-soluble organic solvents are preferred. Preferred agents of the invention are characterized in that they include in addition a nonaqueous solvent, preferred agents of the invention including the solvent in a concentration of 0.1 to 30% by weight, preferably in a concentration of 1 to 20% by weight, very particularly preferably in a concentration of 2 to 10% by weight, based in each case on the agent.

The agents of the invention include at least one oxidation dye precursor as a first component, essential to the invention, in preparation (A). Preferably, the agent includes one or more developer components and optionally one or more coupler components.

Under the influence of oxidizing agents or atmospheric oxygen, the developer components form the actual dyes with one another or during coupling with one or more coupler components. Primary aromatic amines with a further free or substituted hydroxy or amino group, located in the para or ortho position, diaminopyridine derivatives, heterocyclic hydrazones, 4-aminopyrazolone derivatives, and 2,4,5,6-tetraaminopyrimidine and derivatives thereof are typically used as developer components.

It can be preferred according to the invention to use a p-phenylenediamine derivative or one of the physiologically acceptable salts thereof as a developer component.

Particularly preferred are the p-phenylenediamine derivatives of the formula (E1)

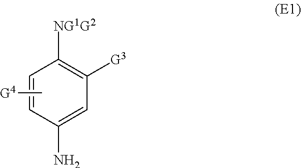

where
G¹ stands for a hydrogen atom, a C₁-C₄ alkyl group, a C₁-C₄ monohydroxyalkyl group, a C₂-C₄ polyhydroxyalkyl group, a C₁-C₄-alkoxy-(C₁-C₄)-alkyl group, a 4-aminophenyl group, or a C₁-C₄ alkyl group, which is substituted with a nitrogen-containing group, a phenyl or a 4-aminophenyl group;
G² stands for a hydrogen atom, a C₁-C₄ alkyl group, a C₁-C₄ monohydroxyalkyl group, a C₂-C₄ polyhydroxyalkyl group, a C₁-C₄-alkoxy-(C₁-C₄)-alkyl group, or a C₁-C₄ alkyl group, which is substituted with a nitrogen-containing group;
G³ stands for a hydrogen atom, a halogen atom, such as a chlorine, bromine, iodine, or fluorine atom, a C₁-C₄ alkyl group, a C₁-C₄ monohydroxyalkyl group, a C₂-C₄ polyhydroxyalkyl group, a C₁-C₄ hydroxyalkoxy group, a C₁-C₄-alkoxy-(C₁-C₄)-alkyl group, a C₁-C₄ acetylaminoalkoxy group, a mesylamino (C₁-C₄) alkoxy group, or a C₁-C₄ carbamoylaminoalkoxy group;
G⁴ stands for a hydrogen atom, a halogen atom, a C₁-C₄ alkyl group, or a C₁-C₄-alkoxy-(C₁-C₄)-alkyl group, or
if G³ and G⁴ are in the ortho position to one another, they can together form a bridging α,ω-alkylenedioxo group such as, for example, an ethylenedioxy group.

Particularly preferred p-phenylenediamines of the formula (E1) are selected from one or more compounds from the group formed by p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis(2-hydroxyethyl)-p-phenylenediamine, 4-N,N-bis(2-hydroxyethyl)amino-2-methylaniline, 4-N,N-bis(2-hydroxyethyl)amino-2-chloroaniline, 2-(2-hydroxyethyl)-p-phenylene-diamine, 2-(1,2-dihydroxyethyl)-p-phenylenediamine, 2-fluoro-p-phenylenediamine, 2-isopropyl-p-phenylenediamine, N-(2-hydroxypropyl)-p-phenylenediamine, 2-hydroxymethyl-p-phenylenediamine, N,N-dimethyl-3-methyl-p-phenylenediamine, N,N-(ethyl, 2-hydroxyethyl)-p-phenylenediamine, N-(2,3-dihydroxypropyl)-p-phenylenediamine, N-(4-aminophenyl)-p-phenylenediamine, N-phenyl-p-phenylene-diamine, 2-(2-hydroxyethyloxy)-p-phenylenediamine, 2-methoxymethyl-p-phenylenediamine, 2-(2-acetylaminoethyloxy)-p-phenylenediamine, N-(2-methoxyethyl)-p-phenylenediamine, N-(4-amino-3-methylphenyl)-N-[3-(1H-imidazol-1-yl)propyl]amine, 5,8-diaminobenzo-1,4-dioxane, and the physiologically acceptable salts thereof. p-Phenylenediamine derivatives of the formula (E1) very particularly preferred according to the invention are selected from at least one compound from the group comprising p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, N-(4-amino-3-methylphenyl)-N-[3-(1H-imidazol-1-yl)propyl]amine, 2-methoxymethyl-p-phenylenediamine, and the physiologically acceptable salts thereof.

It can be preferable furthermore according to the invention to use as the developer component compounds that include at least two aromatic rings substituted with amino and/or hydroxyl groups.

Among bicyclic developer components that can be used in the dye compositions according to the invention, compounds can be named in particular that correspond to the following formula (E2) and the physiologically acceptable salts thereof,

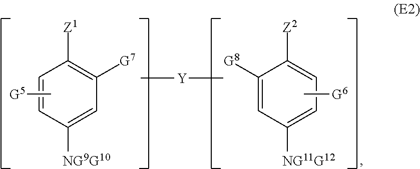

where
Z¹ and Z² independently of one another stand for a hydroxy or NH₂ group, which is optionally substituted by a C₁-C₄ alkyl group, by a C₁-C₄ hydroxyalkyl group, and/or by a bridge Y or which optionally is part of a bridging ring system,
the bridge Y stands for an alkylene group having 1 to 14 carbon atoms, such as a linear or branched alkylene chain or an alkylene ring which can be interrupted or terminated by one or more nitrogen-containing groups and/or one or more heteroatoms, such as oxygen, sulfur, or nitrogen atoms, and may possibly be substituted by one or more hydroxyl or C₁-C₈ alkoxy groups, or for a direct bond,
G⁵ and G⁶ independently of one another stand for a hydrogen or halogen atom, a C₁-C₄ alkyl group, a C₁-C₄ monohydroxyalkyl group, a C₂-C₄ polyhydroxyalkyl group, a C₁-C₄ aminoalkyl group, or a direct bond to bridge Y,
G⁷, G⁸, G⁹, G¹⁰, G¹¹, and G¹² independently of one another stand for a hydrogen atom, a direct bond to bridge Y, or a C₁-C₄ alkyl group,
   with the proviso that the compounds of the formula (E2) include only one bridge Y per molecule.

Preferred bicyclic developer components of the formula (E2) are selected in particular from at least one of the following compounds: N,N′-bis(2-hydroxyethyl)-N,N′-bis(4′-aminophenyl)-1,3-diaminopropan-2-ol, N,N′-bis(2-hydroxyethyl)-N,N′-bis(4-aminophenyl)ethylenediamine, N,N′-bis(4-aminophenyl)tetramethylenediamine, N,N′-bis(2-hydroxyethyl)-N,N′-bis(4-aminophenyl)tetramethylenediamine, N,N′-bis(4-(methylamino)phenyl)tetramethylenediamine, N,N′-diethyl-N,N′-bis(4-amino-3-methylphenyl)ethylenediamine, bis(2-hydroxy-5-aminophenyl)methane, N,N′-bis(4-aminophenyl)-1,4-diazacycloheptane, N,N′-bis(2-hydroxy-5-aminobenzyl)piperazine, N-(4-aminophenyl)-p-phenylenediamine, and 1,10-bis(2′,5′-diaminophenyl)-1,4,7,10-tetraoxadecane, and the physiologically acceptable salts thereof. Very especially preferred bicyclic developer components of the formula (2) are selected from among N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxa-decane, or one of the physiologically acceptable salts thereof.

It can be preferred furthermore according to the invention to use a p-aminophenol derivative or one of the physiologically acceptable salts thereof as a developer component.

Particularly preferred are the p-aminophenol derivatives of the formula (E3),

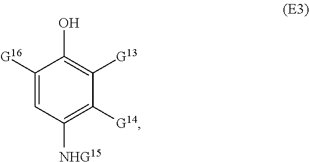

where
G¹³ stands for a hydrogen atom, a halogen atom, a C₁-C₄ alkyl group, a C₁-C₄ monohydroxyalkyl group, a C₂-C₄ polyhydroxyalkyl group, a C₁-C₄-alkoxy-(C₁-C₄)-alkyl group, a C₁-C₄ aminoalkyl group, a hydroxy-(C₁-C₄)-alkylamino group, a C₁-C₄ hydroxyalkoxy group, a C₁-C₄-hydroxyalkyl-(C₁-C₄)-aminoalkyl group, or a di-[C₁-C₄)-alkyl]amino-(C₁-C₄)-alkyl group, and
G¹⁴ stands for a hydrogen or halogen atom, a C₁-C₄ alkyl group, a C₁-C₄ monohydroxyalkyl group, a C₂-C₄ polyhydroxyalkyl group, a C₁-C₄-alkoxy-(C₁-C₄)-alkyl group, a C₁-C₄ aminoalkyl group, or a C₁-C₄ cyanoalkyl group,
G¹⁵ stands for hydrogen, a C₁-C₄ alkyl group, a C₁-C₄ monohydroxyalkyl group, a C₂-C₄ polyhydroxyalkyl group, a phenyl group, or a benzyl group, and
G¹⁶ stands for hydrogen or a halogen atom.

Preferred p-aminophenols of the formula (E3) are particularly p-aminophenol, N-methyl-p-aminophenol, 4-amino-3-methylphenol, 4-amino-3-fluorophenyl, 2-hydroxymethylamino-4-aminophenol, 4-amino-3-hydroxymethylphenol, 4-amino-2-(β-hydroxyethyl)phenol, 4-amino-2-methylphenol, 4-amino-2-hydroxymethylphenol, 4-amino-2-methoxymethylphenol, 4-amino-2-aminomethylphenol, 4-amino-2-(2-hydroxyethylaminomethyl)phenol, 4-amino-2-(1,2-dihydroxyethyl)phenol, 4-amino-2-fluorophenol, 4-amino-2-chlorophenol, 4-amino-2,6-dichlorophenol, 4-amino-2-(diethylaminomethyl)phenol, and the physiologically acceptable salts thereof. Particularly preferred compounds of the formula (E3) are p-aminophenol, 4-amino-3-methylphenol, 4-amino-2-aminomethylphenol, 4-amino-2-(1,2-dihydroxyethyl)phenol, and 4-amino-2-(diethylaminomethyl)phenol.

Further, the developer component can be selected from o-aminophenol and derivatives thereof, such as, for example, 2-amino-4-methylphenol, 2-amino-5-methylphenol, or 2-amino-4-chlorophenol.

Furthermore, the developer component can be selected from heterocyclic developer components, such as, for example, pyrimidine derivatives, pyrazole derivatives, pyrazolopyrimidine derivatives, or the physiologically acceptable salts thereof.

Particularly preferred pyrimidine derivatives are in particular the compounds: 2,4,5,6-tetraaminopyrimidine, 4-hydroxy-2,5,6-triaminopyrimidine, 2-hydroxy-4,5,6-triaminopyrimidine, 2-dimethylamino-4, 5,6-triaminopyrimidine, 2,4-dihydroxy-5,6-diaminopyrimidine, and 2,5,6-triaminopyrimidine. Particularly preferred pyrazole derivatives are in particular the compounds, selected from among 4,5-diamino-1-methylpyrazole, 4,5-diamino-1-(3-hydroxyethyl)pyrazole, 3,4-diaminopyrazole, 4,5-diamino-1-(4-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4, 5-diamino-1-tert-butyl-3-methylpyrazole, 4, 5-diamino-1-(β-hydroxyethyl)-3-methylpyrazole, 4,5-diamino-1-ethyl-3-methylpyrazole, 4,5-diamino-1-ethyl-3-(4-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2-aminoethyl)amino-1,3-dimethylpyrazole, and the physiologically acceptable salts thereof.

Preferred pyrazolopyrimidines are pyrazolo[1,5-a]pyrimidines. Particularly preferred pyrazolo[1,5-a]pyrimidines are again pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1, 5-a]pyrimidin-7-yl)-(2-hydroxyethyl)amino]ethanol, 2-[(7-aminopyrazolo[1,5a]pyrimidin-3-yl)-(2-hydroxyethyl)amino]ethanol, 5,6-dimethyl-pyrazolo[1,5-a]pyrimidin-3,7-diamine, 2,6-dimethylpyrazolo[1,5-a]pyrimidine-3,7-diamine, 3-amino-7-dimethylamino-2,5-dimethylpyrazolo[1,5-a]pyrimidine, and the physiologically acceptable salts thereof and the tautomeric forms thereof, if a tautomeric equilibrium exists.

Very particularly preferred developer components are selected from at least one compound from the group, formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, N-(4-amino-3-methylphenyl)-N-[3-(1H-imidazol-1-yl)-propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2, 5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, and the physiologically acceptable salts thereof.

The developer components are preferably used in an amount of 0.005 to 20% by weight, preferably 0.1 to 5% by weight, in each case based on the ready-to-use oxidation dye.

Coupler components during oxidative dyeing alone cause no significant coloring, but always require the presence of developer components. Therefore, it is preferred according to the invention that at least one coupler component is used in addition when at least one developer component is employed.

Coupler components in the context of the invention permit at least one substitution of a chemical group of the coupler by the oxidized form of the developer component. In this regard, a covalent bond forms between the coupler and developer component. Couplers are preferably cyclic compounds, which have at least two groups on the ring, selected from (i) optionally substituted amino groups and/or (ii) hydroxy groups. These groups are conjugated by a double-bond system.

Coupler components of the invention are preferably selected from the classes of m-aminophenol and/or derivatives thereof, m-diaminobenzene and/or derivatives thereof, o-diaminobenzene and/or derivatives thereof, naphthalene derivatives with at least one hydroxy group, di- or trihydroxybenzene and/or derivatives thereof, pyridine derivatives, pyrimidine derivatives, monohydroxyindole derivatives and/or monoaminoindole derivatives, monohydroxyindoline derivatives and/or monoaminoindoline derivatives, pyrazolone derivatives, such as 1-phenyl-3-methylpyrazol-5-one, morpholine derivatives, such as 6-hydroxybenzomorpholine or 6-aminobenzomorpholine, quinoxaline derivatives, such as 6-methyl-1,2,3,4-tetrahydroquinoxaline, and mixtures of two or more compounds from one or more of these classes.

Preferred m-aminophenol coupler components are selected from at least one compound from the group, formed by m-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, 2,4-dichloro-3-aminophenol, and the physiologically acceptable salts thereof.

Preferred m-diaminobenzene coupler components are selected from at least one compound from the group, formed by 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2, 6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4, 5-dimethylphenyl}amino)ethanol, 2-[3-morpholin-4-ylphenyl)-amino]ethanol, 3-amino-4-(2-methoxyethoxy)-5-methylphenylamine, 1-amino-3-bis(2′-hydroxyethyl)aminobenzene, and the physiologically acceptable salts thereof.

Preferred o-diaminobenzene coupler components are selected from at least one compound from the group formed by 3,4-diaminobenzoic acid and 2,3-diamino-1-methylbenzene, and the physiologically acceptable salts thereof.

Preferred di- or trihydroxybenzenes and derivatives thereof are selected from at least one compound from the group formed by resorcinol, resorcinol monomethyl ether, 2-methylresorcinol, 5-methylresorcinol, 2,5-dimethylresorcinol, 2-chlororesorcinol, 4-chlororesorcinol, pyrogallol, and 1,2,4-trihydroxybenzene.

Preferred pyridine derivatives are selected from at least one compound from the group formed by 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 2,6-dihydroxy-3,4-dimethylpyridine, 3,4-diaminopyridine, 2-(2-methoxyethyl)amino-3-amino-6-methoxypyridine, 2-(4′-methoxyphenyl)amino-3-aminopyridine, and the physiologically acceptable salts thereof.

Preferred naphthalene derivatives with at least one hydroxy group are selected from at least one compound from the group formed by 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene, and 2,3-dihydroxynaphthalene.

Preferred indole derivatives are selected from at least one compound from the group formed by 4-hydroxyindole, 6-hydroxyindole, and 7-hydroxyindole, and the physiologically acceptable salts thereof.

Preferred indoline derivatives are selected from at least one compound from the group formed by 4-hydroxyindoline, 6-hydroxyindoline, and 7-hydroxyindoline, and the physiologically acceptable salts thereof.

Preferred pyrimidine derivatives are selected from at least one compound from the group formed by 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, and the physiologically acceptable salts thereof.

Particularly preferred coupler components according to the invention are selected from among m-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, 2,4-dichloro-3-aminophenol, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, resorcinol monomethyl ether, 2-methylresorcinol, 5-methylresorcinol, 2,5-dimethylresorcinol, 2-chlororesorcinol, 4-chlororesorcinol, pyrogallol, 1,2,4-trihydroxybenzene, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 2,6-dihydroxy-3,4-dimethylpyridine, 3,4-diaminopyridine, 2-(2-methoxyethyl)amino-3-amino-6-methoxy-pyridine, 2-(4′-methoxyphenyl)amino-3-aminopyridine, 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1, 8-dihydroxynaphthalene, 2,7-dihydroxynaphthalene, 2,3-dihydroxynaphthalene, 4-hydroxyindole, 6-hydroxyindole, 7-hydroxyindole, 4-hydroxyindoline, 6-hydroxyindoline, 7-hydroxyindoline, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, or mixtures of said compounds or the physiologically acceptable salts thereof.

The coupler components are preferably used in an amount of 0.005 to 20% by weight, preferably 0.1 to 5% by weight, in each case based on the ready-to-use oxidation dye.

In this case, the developer components and coupler components are generally used in approximately molar amounts to one another. Although molar use has proven to be expedient, a certain excess of individual oxidation dye precursors is not disadvantageous, so that developer components and coupler components may have a molar ratio of 1:0.5 to 1:3, particularly 1:1 to 1:2.

In order to provide further nuances of the resulting color shades, it can be preferred according to the invention further to add at least one direct dye to the agent. These are dye molecules that are directly absorbed onto the substrate and do not require any oxidative process to develop the color. These dyes include, for example, henna which was already known in antiquity for dyeing skin and hair. Today direct dyes are usually nitrophenylenediamines, nitroaminophenols, azo dyes, anthraquinones, or indophenols.

The direct dyes are each preferably used in an amount of 0.001 to 20% by weight, based on the total application preparation. The total amount of direct dyes is preferably at most 20% by weight. Direct dyes can be divided into anionic, cationic, and nonionic direct dyes.

Preferred anionic direct dyes are the compounds known under the international names or trade names: Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, tetrabromophenol blue, and bromophenol blue.

Preferred cationic direct dyes in this context are:
(a) cationic triphenylmethane dyes such as, for example, Basic Blue 7, Basic Blue 26, Basic Violet 2, and Basic Violet 14,
(b) aromatic systems substituted by a quaternary nitrogen group such as, for example, Basic Yellow 57, Basic Red 76, Basic Blue 99, Basic Brown 16, and Basic Brown 17, and
(c) direct dyes including a heterocycle, which has at least one quaternary nitrogen atom, as specified, for example, in claims 6 to 11 in EP-A2-998 908, expressly incorporated here by reference. The compounds, which are also known under the names: Basic Yellow 87, Basic Orange 31, and Basic Red 51, are very particularly preferred cationic direct dyes.

The cationic direct dyes, which are marketed under the trademark Arianor®, are cationic direct dyes also very particularly preferred according to the invention.

Preferred nonionic direct dyes are compounds known under the international names or trade names: HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, and Disperse Black 9, and 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

It is not required that the direct dyes each represent pure compounds. Rather, due to the production methods for the individual dyes, minor amounts of other components can be present, provided these have no detrimental effect on the coloring result or must be excluded for other reasons, e.g., toxicological.

Furthermore, naturally occurring dyes can also be used as direct dyes, as are found, for example, in henna red, henna neutral, henna black, chamomile blossoms, sandalwood, black tea, buckthorn bark, sage, logwood, madder root, catechu, and alkanna root.

It has proven advantageous, furthermore, if to provide additional nuances, the agents of the invention include in addition one or more dye precursors of nature-analogous dyes. The dye precursors of nature-analogous dyes that are used are preferably indoles and indolines which have at least two groups selected from hydroxy and/or amino groups, preferably as substituents on the six-membered ring. These groups can carry further substituents, e.g., in the form of an etherification or esterification of the hydroxy group or alkylation of the amino group. In a further embodiment, the dyes include at least one indole and/or indoline derivative. Compositions of the invention that include the precursors of nature-analogous dyes are used preferably as air-oxidative dyes. In this embodiment, said compositions consequently are not combined with an additional oxidizing agent.

Derivatives of 5,6-dihydroxyindoline are especially well suited as precursors of nature-analogous hair dyes. Particularly preferred indoline derivatives are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, and 5,6-dihydroxyindoline-2-carboxylic acid. Derivatives of 5,6-dihydroxyindole are exceptionally suitable, furthermore, as precursors of nature-analogous hair dyes. Preferred indole derivatives are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, and 5,6-dihydroxyindole-2-carboxylic acid.

The agents of the invention include at least one alkali metal salts of at least one fatty acid as a second component, essential to the invention, in preparation (A).

Particularly preferred in this case are the sodium salts and/or the potassium salts, the potassium salts being extremely preferred.

Especially suitable according to the invention are the potassium salts of the following fatty acids: caproic acid (hexanoic acid), enanthic acid (heptanoic acid), caprylic acid (octanoic acid), pelargonic acid (nonanoic acid), capric acid (decanoic acid), lauric acid (dodecanoic acid), myristic acid (tetradecanoic acid), palmitic acid (hexadecanoic acid), margaric acid (heptadecanoic acid), stearic acid (octadecanoic acid), arachidic acid (eicosanoic/icosanoic acid), behenic acid (docosanoic acid), lignoceric acid (tetracosanoic acid), cerotic acid (hexacosanoic acid), montanic acid (octacosanoic acid), melissic acid (triacontanoic acid), undecylenic acid ((10Z)-undec-10-enoic acid), myristoleic acid ((9Z)-tetradec-9-enoic acid), palmitoleic acid ((9Z)-hexadec-9-enoic acid), petroselinic acid ((6Z)-octadec-6-enoic acid), oleic acid ((9Z)-octadec-9-enoic acid), elaidic acid ((9E)-octadec-9-enoic acid), vaccenic acid ((11E)-octadec-11-enoic acid), gadoleic acid ((9Z)-eicos-9-enoic acid), eicosenoic acid ((11Z)-eicos-11-enoic acid), cetoleic acid ((11Z)-docos-11-enoic acid), erucic acid ((13Z)-docos-13-enoic acid), nervonic acid ((15Z)-tetracos-15-enoic acid), linoleic acid ((9Z,12Z)-octadeca-9,12-dienoic acid), alpha-linolenic acid ((9Z,12Z,15Z)-octadeca-9,12,15-trienoic acid), gamma-linolenic acid ((6Z,9Z,12Z)-octadeca-6,9,12-trienoic acid), calendic acid ((8E,10E,12Z)-octadeca-8,10,12-trienoic acid), punicic acid ((9Z,11E,13Z)-octadeca-9,11,13-trienoic acid), alpha-elaeostearic acid ((9Z,11E,13E)-octadeca-9,11,13-trienoic acid), beta-elaeostearic acid ((9E,11E,13E)-octadeca-9,11,13-trienoic acid), arachidonic acid ((5Z,8Z,11Z,14Z)-eicosa-5,8,11,14-tetraenoic acid), timnodonic acid ((5Z,8Z,11Z,14Z,17Z)-eicosa-5,8,11,14,17-pentaenoic acid), clupanodonic acid ((7Z,10Z,13Z,16Z,19Z)-docosa-7,10,13,16,19-pentaenoic acid), and cervonic acid ((4Z,7Z,10Z,13Z,16Z,19Z)-docosa-4,7,10,13,16,19-hexaenoic acid).

Particularly preferred agents of the invention are characterized in that preparation (A) includes one or more of the following potassium salts: potassium caproate, potassium enanthate, potassium caprylate, potassium pelargonate, potassium caprate, potassium laurate, potassium myristate, potassium palmitate, potassium margarate, potassium stearate, potassium arachidate, potassium behenate, potassium lignocerate, potassium cerotate, potassium montanate, potassium melissate, potassium undecylenate, potassium myristoleate, potassium palmitoleate, potassium petroselinate, potassium oleate, potassium elaidate, potassium vaccenate, potassium gadoleate, potassium eicosenoate, potassium cetoleate, potassium erucate, potassium nervonate, potassium linoleate, potassium alpha-linolenate, potassium gamma-linolenate, potassium calendate, potassium punicate, potassium alpha-elaeostearate, potassium beta-elaeostearate, potassium arachidonate, potassium timnodonate, potassium clupanodonate, and potassium cervonate.

Very particularly preferred agents of the invention include at least one potassium salt of at least one unsaturated fatty acid in preparation (A). Here, preferred agents of the invention are characterized in that preparation (A) includes potassium palmitoleate and/or potassium petroselinate and/or potassium oleate and/or potassium gadoleate and/or potassium eicosenoate and/or potassium cetoleate and/or potassium erucate and/or potassium nervonate and/or potassium linoleate and/or potassium alpha-linolenate and/or potassium gamma-linolenate and/or potassium calendate.

Regardless of whether one or more potassium salts are used, agents and methods particularly preferred according to the invention are characterized in that preparation (A), based on its weight, includes 0.1 to 10% by weight, preferably 0.15 to 7.5% by weight, more preferably 0.2 to 6% by weight, particularly preferably 0.25 to 5.5% by weight, and in particular 0.3 to 5% by weight of at least one potassium salt of at least one fatty acid.

Extremely preferred agents of the invention include only potassium salts of fatty acids and are free of sodium salts of fatty acids.

Preparations (A) are preferably alkaline, particularly preferred agents and methods of the invention being characterized in that preparation (A) has a pH of 8 to 12, preferably of 8.5 to 11.5, and particularly of 9 to 11.

Preferably at least one alkanolamine is used as an alkalizing agent in this case.

Alkanolamines that can be used according to the invention are preferably selected from alkanolamines from primary, secondary, or tertiary amines with a C₂-C₆ alkyl parent structure, bearing at least one hydroxyl group. Particularly preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, methylglucamine, triethanolamine, diethanolamine, and triisopropanolamine.

Alkanolamines very especially preferred according to the invention are selected from the group comprising 2-aminoethan-1-ol (monoethanolamine), 2-amino-2-methylpropan-1-ol, 2-amino-2-methylpropane-1,3-diol, and triethanolamine. Particularly preferred agents include at least monoethanolamine as the alkanolamine.

Preferably, the alkanolamines are present in an amount of 0.05 to 20% by weight, particularly of 0.5 to 15% by weight, based in each case on the total weight of the ready-to-use agent.

Particularly preferred agents and methods of the invention are characterized in that preparation (A), based on its weight, includes 0.1 to 15% by weight, preferably 0.5 to 10% by weight, more preferably 2 to 9% by weight, particularly preferably 3 to 8% by weight, and in particular 4 to 7% by weight of at least one alkanolamine from primary, secondary, or tertiary amines with a C₂-C₆ alkyl parent structure, bearing at least one hydroxyl group, preferably selected from the group, formed by 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, triethanolamine, diethanolamine, and triisopropanolamine, and particularly preferably selected from monoethanolamine.

Preparation (A), moreover, can include further ingredients. These are described below.

The agents and methods of the invention are characterized further in that preparation (B) includes at least one oxidizing agent.

In a particular embodiment, preparations (B) according to the invention include hydrogen peroxide as the oxidizing agent.

The concentration of a hydrogen peroxide solution in the oxidizing agent preparation (B) is determined, on the one hand, by legal requirements and, on the other, by the desired effect. Preferably, preparations (B) include, based on their weight, hydrogen peroxide (calculated as 100% H₂O₂) in amounts of 0.1 to 30% by weight, preferably of 0.5 to 20% by weight, particularly preferably of 1 to 15% by weight, and very particularly preferably of 3 to 8% by weight.

Particularly preferred agents or methods of the invention are characterized in that preparation (B), based on its weight, includes 0.1 to 15% by weight, preferably 0.25 to 12% by weight, more preferably 0.5 to 10% by weight, and in particular 3 to 8% by weight of hydrogen peroxide (calculated as 100% H₂O₂).

Ready-to-use agents preferred according to the invention are characterized in that, based on the total weight of the ready-to-use agent, they include 0.01 to 12% by weight, preferably 0.1 to 10% by weight, particularly preferably 1 to 8% by weight of hydrogen peroxide.

To stabilize the hydrogen peroxide, the pH of preparation (B) can be adjusted preferably to pH 3 to 5, particularly preferably to pH 3.5 to 4.5, and very particularly preferably to pH 3.8 to 4.2.

Particularly preferred agents or methods of the invention are characterized in that preparation (B) has a pH of 3 to 5, particularly preferably of pH 3.5 to 4.5, and very particularly preferably of pH 3.8 to 4.2.

Preparation (B) includes xanthan gum as the second essential component.

The use of the xanthan gum biopolymer, sold under the trade name Keltrol CG-SFT from the company Kelco, is particularly preferred. Other suitable commercial xanthan gums are, for example, Kelzan (xanthan gum biopolymer, Kelco), Xanthan FN (xanthan gum biopolymer, Jungbunzlauer), Keltrol, e.g., Keltrol CG-T (xanthan gum biopolymer, Kelco), or the aforesaid Keltrol CG-SFT (xanthan gum biopolymer, Kelco).

Agents or methods particularly preferred according to the invention are characterized in that preparation (B), based on its weight, includes 0.01 to 4% by weight, preferably 0.02 to 3.5% by weight, more preferably 0.03 to 3% by weight, particularly preferably 0.04 to 2.5% by weight, and in particular 0.05 to 2% by weight of xanthan gum.

Agents or methods particularly preferred according to the invention are characterized in that preparation (B), based on its weight, includes 0.01 to 4% by weight, preferably 0.02 to 3.5% by weight, more preferably 0.03 to 3% by weight, particularly preferably 0.04 to 2.5% by weight, and in particular 0.05 to 2% by weight of xanthan gum.

Both preparation (A) (see above) and preparation (B) may include further ingredients, surfactant(s) and care substance(s) being particularly important.

A surface-active substance is preferably added in addition to preparations (A) and (B), such surface-active substances being called surfactants or emulsifiers depending on the field of application: they are preferably selected from anionic, cationic, zwitterionic, amphoteric, and nonionic surfactants and emulsifiers.

A surface-active substance is preferably added in addition to preparations (A) and (B), such surface-active substances being called surfactants or emulsifiers depending on the field of application: they are preferably selected from zwitterionic and nonionic surfactants and emulsifiers.

Agents preferred according to the invention are characterized in that the agent includes in addition at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethylimidazolines. A preferred zwitterionic surfactant is known by the INCI name Cocamidopropyl Betaine.

It has proven advantageous, furthermore, for the agents to include other non-ionogenic surface-active substances. Preferred nonionic surfactants are, for example, alkylene oxide adducts to fatty alcohols and fatty acids each with 2 to 30 mol of ethylene oxide per mole of fatty alcohol or fatty acid. Preparations with excellent properties are likewise obtained if they include fatty acid esters of ethoxylated glycerol as the nonionic surfactants.

Alkyl polyglycosides are particularly preferred. The combination of alkyl polyglycosides and zwitterionic surfactants is very particularly preferred.

Especially preferred nonionic surfactants in this regard are alkylene oxide adducts to fatty alcohols and fatty acids each with 2 to 30 mol of ethylene oxide per mole of C₁₂-C₃₀ fatty alcohol or C₁₂-C₃₀ fatty acid. Alkyl polyglycosides are also especially preferred nonionic surfactants.

The nonionic and/or zwitterionic surfactants can be used in proportions of 0.1 to 40% by weight, preferably 1 to 30% by weight, and very particularly preferably of 20 to 25% by weight, based in each case on the total amount of preparations (A) and (b).

Agents and methods particularly preferred according to the invention are characterized in that the application mixture, based on its weight, has a total surfactant content of 1.0 to 50% by weight, preferably of 2.5 to 45% by weight, and in particular of 5 to 40% by weight.

Preferred fatty components in this regard are understood to be components from the group of C₁₂-C₃₀ fatty alcohols, C₁₂-C₃₀ fatty acid triglycerides, C₁₂-C₃₀ fatty acid monoglycerides, and C₁₂-C₃₀ fatty acid diglycerides. Only nonionic substances are regarded explicitly as fatty components in the context of the present invention. Charged compounds such as, for example, fatty acids and salts thereof are not understood to be a fatty component.

C₁₂-C₃₀ fatty alcohols can be saturated, mono- or polyunsaturated, linear or branched fatty alcohols having 12 to 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachidyl alcohol (eicosan-1-ol), heneicosyl alcohol (heneicosan-1-ol), and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

Preferred representatives of branched fatty alcohols are 2-octyldodecanol, 2-hexyldodecanol, and/or 2-butyldodecanol.

A C₁₂-C₃₀ fatty acid triglyceride in the context of the present invention is understood to mean the triester of the trihydric alcohol, glycerol, with three equivalents of fatty acids. In this regard, both structurally similar and also different fatty acids can be involved in ester formations within a triglyceride molecule.

It is possible that preparation (A) and/or preparation (B) include fatty substances. Fatty acids according to the invention are understood to mean saturated or unsaturated, unbranched or branched, unsubstituted or substituted C₁₂-C₃₀ carboxylic acids. Unsaturated fatty acids can be mono- or polyunsaturated. In the case of an unsaturated fatty acid, the C—C double bond(s) thereof can have the cis or trans configuration.

Notable for particular suitability are fatty acid triglycerides in which at least one of the ester groups originating from glycerol is formed with a fatty acid, selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides can also be of natural origin. The fatty acid triglycerides, occurring in soybean oil, peanut oil, olive oil, sunflower oil, macadamia nut oil, Moringa oil, apricot kernel oil, Marula oil, and/or optionally hydrogenated castor oil, or mixtures thereof are especially suitable for use in the product of the invention.

A C₁₂-C₃₀ fatty acid monoglyceride is understood to mean the monoester of the trihydric alcohol, glycerol, with a fatty acid equivalent. In this case, either the middle hydroxy group of glycerol or the terminal hydroxy group of glycerol can be esterified with the fatty acid.

Notable for particular suitability are C₁₂-C₃₀ fatty acid monoglycerides in which a hydroxy group of glycerol is esterified with a fatty acid, the fatty acids being selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], or nervonic acid [(15Z)-tetracos-15-enoic acid].

A C₁₂-C₃₀ fatty acid diglyceride is understood to mean the diester of the trihydric alcohol, glycerol, with two fatty acid equivalents. In this case, either the middle and a terminal hydroxy group of glycerol can be esterified with two fatty acid equivalents, or however both terminal hydroxy groups of glycerol are each esterified with one fatty acid. Glycerol can be esterified hereby both with two structurally similar and with two different fatty acids.

Notable for particular suitability are fatty acid diglycerides in which at least one of the ester groups originating from glycerol is formed with a fatty acid, selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The ready-to-use dyes can include other auxiliary substances and additives. Thus, it has proven advantageous for the agents to include at least one additional thickener. There are no basic restrictions with regard to these thickeners. Both organic and purely inorganic thickeners may be used.

Suitable thickeners are anionic, synthetic polymers; cationic, synthetic polymers; naturally occurring thickeners, such as nonionic guar gums, scleroglucan gums or xanthan gums, gum arabic, gum ghatti, karaya gum, tragacanth gum, carrageenan gum, agar-agar, locust bean flour, pectins, alginates, starch fractions, and derivatives such as amylose, amylopectin, and dextrins, as well as cellulose derivatives such as, for example, methylcellulose, carboxyalkylcelluloses, and hydroxyalkylcelluloses; nonionic, synthetic polymers such polyvinyl alcohol or polyvinylpyrrolidinone; as well as inorganic thickeners, in particular phyllosilicates such as, for example, bentonite, in particular smectites, such as montmorillonite or hectorite.

The agents of the invention may also include zwitterionic polymers. Preferred zwitterionic polymers are selected from the group comprising:
copolymers of dimethyldiallylammonium salts and acrylic acid, e.g., Polyquaternium-22,
copolymers of dimethyldiallylammonium salts and methacrylic acid,
copolymers of N,N,N-trimethyl-3-[(2-methyl-1-oxo-2-propen-1-yl)amino]-1-propanaminium salts and acrylic acid,
copolymers of N,N,N-trimethyl-3-[(2-methyl-1-oxo-2-propen-1-yl)amino]-1-propanaminium salts and methacrylic acid,
copolymers of N,N,N-trimethyl-2-[(2-methyl-1-oxo-2-propen-1-yl)amino]-1-ethanaminium salts and acrylic acid,
copolymers of N,N,N-trimethyl-2-[(2-methyl-1-oxo-2-propen-1-yl)amino]-1-ethanaminium salts and methacrylic acid,
copolymers of N,N,N-trimethyl-3-[(2-methyl-1-oxo-2-propen-1-yl)amino]-1-propanaminium salts, acrylic acid, and acrylamide, e.g., Polyquaternium-53,
copolymers of N,N,N-trimethyl-3-[(2-methyl-1-oxo-2-propen-1-yl)amino]-1-propanaminium salts, methacrylic acid, and acrylamide,
copolymers of 1-ethenyl-3-methyl-1H-imidazolium salts, 1-ethenyl-1H-imidazole, 1-ethenyl-2-pyrrolidinone, and methacrylic acid, e.g., Polyquaternium-86,
copolymers of 1-ethenyl-3-methyl-1H-imidazolium salts, 1-ethenyl-1H-imidazole, 1-ethenyl-2-pyrrolidinone, and acrylic acid.

The agents of the invention may also include mixtures of the aforementioned preferred zwitterionic polymers (c).

Preparations (A) and (B) furthermore can include additional active substances, auxiliary substances, and additives in order to improve the coloring or lightening performance and to set other desired properties of the agents.

It has proven advantageous, if the coloring agents (i.e., preparations (A) and/or (b)), particularly if they include hydrogen peroxide in addition, include at least one stabilizer or complexing agent. Especially preferred stabilizers are phenacetin, alkali benzoates (sodium benzoate), and salicylic acid and dipicolinic acid. Furthermore, all complexing agents in the state of the art can be used. Complexing agents preferred according to the invention are nitrogen-containing polycarboxylic acids, particularly EDTA and EDDS, and phosphonates, particularly 1-hydroxyethane-1,1-diphosphonate (HEDP), and/or ethylenediamine tetramethylene phosphonate (EDTMP), and/or diethylenetriamine pentamethylene phosphonate (DTPMP), or sodium salts thereof.

All ionic complexing agents included in preparations (A) and (B) fall in the group of organic salts, and their employed concentrations must therefore be considered in calculating the total molalities M1 or M2.

Further, the agents of the invention can include other active substances, auxiliary substances, and additives, such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxyl, alkoxy, and/or hydroxyl groups (dimethicone copolyols), linear polysiloxanes(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; pigments and propellants such as propane-butane mixtures, N₂O, dimethyl ether, CO₂, and air.

The selection of these additional substances is made by the skilled artisan according to the desired properties of the agents. In regard to other facultative components and the employed amounts of said components, reference is made expressly to relevant handbooks known to the skilled artisan. The additional active and auxiliary substances are used in the agents of the invention preferably in each case in amounts of 0.0001 to 25% by weight, in particular of 0.0005 to 15% by weight, based on the total weight of the application mixture.

As already mentioned several times, preparations (A) and (B) are mixed to form an application mixture, which was also called the agent of the invention. Although preparations (A) and (B) can be mixed together in any ratio, it has emerged as preferable to use the amounts of preparations (A) and (B) in the application mixture in approximately identical weights if possible. Agents and methods particularly preferred according to the invention are characterized in that preparation (A) and preparation (B) are mixed in the weight ratio of 2:1 to 1:2, preferably 15:10 to 10:15, and in particular 1:1 to form an application mixture.

It is preferred according to the invention, if the described hair dye preparation is taken up in a suitable dispenser and dispensed for the specific use. In this regard, the hair dye preparation is dispensed basically in the form of a foam. The foam consistency of the preparation is to be understood very broadly in this context and includes any mixture of a flowable preparation and a gaseous component. In this respect, both flowable and substantially solid, stable foam consistencies are included in the subject matter of the invention.

Basically a dispenser of the invention includes at least one reservoir to receive at least one component of the hair dye preparation and an application device to dispense the hair dye preparation in the form of foam. Here the reservoir is especially designed as a tube-shaped or bottle-shaped container, whereas the application device closes this container which is open on one side. The actual dispensing of the preparation is preferably effected by a suitable pressure source that is integrated into the dispenser, particularly the reservoir, or by a manual pressure build up initiated by the actual user of the hair dye preparation.

As an example of dispensers of the invention with an integrated pressure source, pressure vessels can be mentioned that usually have either a suitable pressure accumulator inside the container, e.g., a mechanical one, or include a propellant, and in this way place the inside of the container under pressure. These types of pressure vessels usually have suitable valve devices for dispensing the preparation located inside the pressure vessel during an appropriate valve actuation. Such pressure vessels in conjunction with gaseous and/or liquid propellants are mainly known in the form of aerosol dispensers for the most varied cosmetic applications, e.g., hair styling sprays, hair dye preparations, deodorant sprays, shaving foam/gel, etc.

Alternatively, manually actuated dispensers can also be used according to the invention, which rely solely on the force exerted by the user in order to dispense a foamed preparation. In the case of these types, an additional pressure source (e.g., propellant) can be advantageously omitted, which is desirable principally on the grounds of cost and sustainability. Such foam dispensers actuated by manual force provide not only for the delivery of the hair dye preparation from the reservoir to the dispensing outlet, but also for an appropriate foaming of the hair dye preparation. During this foaming or foam formation, the hair dye preparation is basically mixed with a gaseous component, particularly air. Specifically, a foaming device that does this is provided for this purpose.

According to a first variant of a manually actuatable dispenser, it is designed as a shakable dispenser, having at least one reservoir for receiving the hair dye preparation, and an associated dispensing device for dispensing the foamed hair dye preparation. In this regard, the dispensing device is connected to the reservoir, particularly detachably. The actual foam formation occurs inside the shakable dispenser by shaking the hair dye preparation inside the reservoir. The shakable dispenser in conjunction with the appropriate movement of the dispenser thereby forms the aforesaid foaming device. Subsequent to this type of foaming, the foamed hair dye preparation can then be dispensed by the dispensing device.

Another reasonable dispenser variant is provided by the design as a squash or squeeze foam dispenser. Such a squeeze foam dispenser possesses, in addition to the at least one reservoir for receiving the hair dye preparation, an appropriate application device inside which the foaming occurs as well as the subsequent delivery of the hair dye preparation. The hair dye preparation is actually delivered from the reservoir by a force exerted onto the flexible wall of the reservoir. Here, the reversible deformation of the reservoir wall creates a pressure increase inside the reservoir, resulting in the hair dye preparation being forced out of the reservoir. To this end, the reservoir wall has to be designed to be sufficiently flexible or reversibly deformable. This is assured by a design thickness of the reservoir wall appropriate to the required application, in conjunction with a suitable choice of material for the reservoir wall. The reservoir wall of a suitable squeeze foam dispenser is preferably made of a polyolefin such as, for example, polypropylene (PP), high density polyethylene (HDPE), medium density polyethylene (MDPE), low density polyethylene (LDPE), and linear low density polyethylene (LLDPE). Among these, polypropylene (PP) is preferred.

The application device of such a squeeze foam dispenser also includes a suitable foaming device for foaming the hair dye preparation. The foaming device is capable of blending a quantity of preparation with a quantity of gas in the appropriate dispensing ratio in order to generate the desired foam consistency of the hair dye preparation. For this purpose, typically a flow of preparation and a flow of gas are drawn together into a mixing chamber of the foaming device and blended together there by fluid dynamic turbulence.

Air is used particularly preferably as the gaseous component for the foam formation and is drawn in either directly from the reservoir or from the surroundings.

Similarly, the dispenser can also be designed as a pump foam dispenser with at least one reservoir for receiving the hair dye preparation as well as an application device, in which case the application device possesses a pump device for delivering both the hair dye preparation and the gaseous component, preferably air, and moreover includes an appropriate foaming device.

When the aforesaid dispenser variants are used in conjunction with multi-component hair dye preparations with mutually incompatible individual components (e.g., multicomponent oxidation hair dye preparations), it must be assured that the individual components are stored separately until the actual application of the preparation. This is advantageously achieved by using a plurality of reservoirs for receiving the respective individual components of the hair dye preparation, each reservoir being in fluid connection with the associated application device in order to deliver the hair dye preparation. As a result, blending of the individual components of the hair dye preparation occurs only immediately before the preparation is dispensed from the dispenser of the invention when the preparation is actually used. For example, a pump foam dispenser of the invention can also be equipped with two or more reservoirs for a plurality of preparation components.

In other respects, a multicomponent hair dye preparation can also be used according to the invention with one of the above-described dispenser variants having only one reservoir and one application device. For this purpose, the reservoir is designed such that it can be opened recloseably. Ideally, the reservoir is closed by the application device, the application device being connected detachably to the reservoir, for example, by a screw or snap connection. This opens up the possibility of prefilling the reservoir even during production with a component of the hair dye preparation and to add additional components of the hair dye preparation into the reservoir only shortly before the actual use of the hair dye preparation. In this connection, the additional components of the hair dye preparation are included in the total hair dye product in the form of a kit within suitable separate containers and are mixed in the reservoir by the user immediately prior to using the hair dye preparation.

Moreover, in addition, one or more porous insert elements can be used in order to positively influence the attainable foam consistency inside the foaming device. Such porous insert elements are, for example, spongy or net-like in structure and are positioned inside the foaming device at a suitable place in the flow channel for the hair dye preparation, for instance, directly upstream of the delivery outlet of the dispenser. The porous insert element therefore allows the flow of the hair dye preparation and as a result of fluid dynamic turbulence affords a finer and more homogenous foam consistency. The foam consistency can therefore be influenced directly depending on the particular design of the porous insert element. When a net-like insert element is used, it has proven expedient to design the net-like insert element preferably with a hole density of 50 to 220 mesh (mesh=number of openings per inch), particularly preferably 90 to 200 mesh, very particularly preferably 125 to 175 mesh. When a plurality of net-like insert elements are used, it is also possible to use insert elements having different hole densities. In this case, the first, upstream-positioned net-like insert element preferably has a hole density of 50 to 220 mesh (mesh=number of openings per inch), particularly preferably 90 to 200 mesh, very particularly preferably 125 to 175 mesh. The second, downstream-positioned net preferably has a hole density of 160 to 280 mesh, particularly 175 to 245 mesh, and very particularly preferably 180 to 220 mesh. Lastly, the number of porous insert elements used as well as their specific hole density or their porosity characteristics can be designed specifically depending on the relevant type of application.

The application temperatures of the resulting foam can be in the range between 15 to 40° C. After a contact time of 5 to 45 minutes, the hair dye is removed from the hair to be colored by rinsing.

The application mixture is applied to the hair as a foam from a suitable vessel and there used generically for coloring the hair. Particularly preferred methods of the invention are characterized in that the dispenser is a single-chamber squeeze foam dispenser or a single-chamber pump foam dispenser, from which the application mixture is applied as a foam, then the thus obtained foam is distributed on the fibers, then remains on the fibers for a period of 1 to 60 minutes, preferably of 5 to 40 minutes, and is then washed out of the fibers.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for coloring keratin fibers includes at least two preparations (A) and (B), packaged separately from one another, and optionally a further preparation (C) packaged separately from (A) and (B), which are mixed immediately before use to form an application mixture, wherein
i preparation (A) includes
a) at least one oxidation dye precursor and
b) at least one alkali metal salt of at least one fatty acid
and
ii preparation (B) is flowable and, based on its weight, includes
c) at least one oxidizing agent and
d) xanthan gum.

2. The agent according to claim 1, wherein preparation (A), based on its weight, includes 0.1 to 10% by weight at least one potassium salt of at least one fatty acid.

3. The agent according to claim 1, wherein preparation (A) has a pH of 8 to 12.

4. The agent according to claim 1, wherein preparation (A), based on its weight, includes 0.1 to 15% by weight at least one alkanolamine selected from the group consisting of 2-aminoethan-1-ol (monoethanolamine), 3-aminopropan-1-ol, 4-aminobutan-1-ol, 5-aminopentan-1-ol, 1-aminopropan-2-ol (monoisopropanolamine), 1-aminobutan-2-ol, 1-aminopentan-2-ol, 1-aminopentan-3-ol, 1-aminopentan-4-ol, 2-amino-2-methylpropanol, 2-amino-2-methylbutanol, 3-amino-2-methylpropan-1-ol, 1-amino-2-methylpropan-2-ol, 3-aminopropane-1,2-diol, 2-amino-2-methylpropane-1,3-diol, 2-amino-2-ethyl-1,3-propanediol, N,N-dimethylethanolamine, triethanolamine, diethanolamine, and triisopropanolamine.

5. The agent according to claim 1, wherein preparation (B), based on its weight, includes 0.01 to 4% by weight xanthan gum.

6. The agent according to claim 1, wherein preparation (B) has a pH of 3 to 5.

7. The agent according to claim 1, wherein preparation (B), based on its weight, includes 0.1 to 15% hydrogen peroxide calculated as 100% H₂O₂.

8. The agent according to claim 1, wherein preparation (A) and preparation (B) are mixed together in the weight ratio of 2:1 to 1:2 to form an application mixture.

9. The agent according to claim 1, wherein the application mixture, based on its weight, has a total surfactant content of 1.0 to 50%.

10. A method for coloring keratin fibers, including
mixing at least two preparations (A) and (B), packaged separately from one another, and optionally a further preparation (C), packaged separately from (A) and (B), immediately before use to form an application mixture, and
applying the mixture from a dispenser in the form of a foam, wherein
i. preparation (A) includes
a) at least one oxidation dye precursor and
b) at least one alkali metal salt of at least one fatty acid
and
ii. preparation (B) is flowable and, based on its weight, includes
a) at least one oxidizing agent and
b) xanthan gum.

11. The method according to claim 10, wherein the dispenser is a single-chamber squeeze foam dispenser or a single-chamber pump foam dispenser, from which the application mixture is applied as a foam, then the thus obtained foam is distributed on the fibers, then remains on the fibers for a period of 1 to 60 minutes, and is then washed out of the fibers.
